(19) United States
(12) Patent Application Publication
(10) Pub. No.: US 2004/0152872 A1
(43) Pub. Date: Aug. 5, 2004

(54) Method for modifying a protein structure

(76) Inventors: Gerd Wohlfahrt, Braunschweig (DE); Harry Boer, Helsinki (FI); Anu Koivula, Helsinki (FI)

Correspondence Address: BIRCH STEWART KOLASCH & BIRCH, PO BOX 747, FALLS CHURCH, VA, 22040-0747, US

(21) Appl. No.: 10/398,799

(22) PCT Filed: Oct. 12, 2001
(86) PCT No.: PCT/FI01/00884

(30) Foreign Application Priority Data
Oct. 13, 2000 (FI) 20002262

(51) Int. Cl. C07K 14/47

(52) U.S. Cl. 530/350; 530/409

(57) ABSTRACT

The present invention relates to stabilisation of proteins. A theoretical basis for new protein engineering strategies is presented, based on defining rules for strong hydrogen bonds in proteins. In the present invention pairs of carboxylic acid side chains are used to stabilise proteins, or to form pH-dependent molecular switches.

### FIELD OF THE INVENTION

The present invention relates to stabilisation of proteins. A theoretical basis for new protein engineering strategies is presented. In the present invention pairs of carboxylic acid side chains are used to stabilise proteins, or to form pH-dependent molecular switches.

### RELATED ART

The biotechnological industry has a long-standing interest in stabilising proteins. Key objectives are stability against temperature, chemical denaturants, oxidation and proteolysis. Nowadays there are two main approaches to accomplish these, directed evolution and rational (structure based) protein design. Understanding of the rules concerning protein stability has been increased, since site-directed mutagenesis studies have provided experimental data on the relative stability of many mutant proteins. These data have confirmed the dominating influence of hydrophobic interactions in the core of a protein structure, but has also revealed the non-negligible role of electrostatic interactions and hydrogen bonds. The side chains of carboxylates (Asp and Glu) as well as of amides (Asn and Gln) are known to have significant roles in stabilising protein structures e.g. by the formation of hydrogen bonds. Altering these amino acids has in many cases been shown to affect protein stability and also enzyme activity, pH dependency and other properties. For example Hodel et al. (1994) and Catanzano et al. (1998) have studied the effect of hydrogen bonds, formed by amidic or carboxylic side chains with the protein backbone, on protein stability.

In the present invention we point out the relevance of a certain type of hydrogen bonds called low barrier hydrogen bonds (LBHB), which are believed to be much stronger than “standard” hydrogen bonds. The proposal that LBHBs play a role in enzymatic catalysis was first put forth in 1993 (see e.g. Gerlt & Gassman, 1993). However, there has been a debate whether or not LBHBs can exist in a condensed phase. In general, the strength of a hydrogen bond depends on its length and linearity, its microenvironment (ligands and solvation) and how well the pKa's of the conjugated acids match. There have been several quantum chemical attempts to characterise the nature of LBHBs. The work of McAllister and co-workers focussed on the formate-formic acid system which could be considered as the simplest model for the interactions of acidic side-chains in proteins. Based on their studies the main determinants for the strength of these LBHBs are: (1) no or asymmetric microsolvation (Pan & McAllister, 1997); (2) geometry (length and angle) (Smallwood & McAllister, 1997); (3) pKa matching (Kumar & McAllister, 1998).

In 1982 Sawyer and James commented on the existence of carboxyl-carboxylate interactions in proteins and their possible importance for protein stability. Since then, in only one systematic approach by Flocco and Mowbray (1995) the occurrence of acid-acid pairs in proteins has been analysed in a subset of the PDB (Brookhaven Protein Data Bank, Bernstein et al., 1977) consisting of proteins having less than 25% amino acid sequence identity. Within 151 protein structures analysed the authors found some tendencies in the spatial arrangement and environmental properties of the 28 occurring acid-acid pairs. Their main findings are: (1) the most common O—O separation is 2.6-2.7 Å; (2) all pairs are involved in additional hydrogen bonds; (3) most pairs with short O—O distances have low solvent accessibilities; (4) the angle between the plane of one carboxyl group and the closest O of the other carboxyl group is close to 90°; (5) there is less preference for the “out-out” (anti-anti) arrangement. The authors suggest that these types of acid pairs might be important in enzyme catalysis and binding of the substrate.

On the other hand, Mortensen and Breddam showed that the stability of a serine carboxypeptidase can be significantly decreased by removing a pair of glutamic acids in the active centre of the enzyme (Mortensen & Breddam, 1994). The destabilising effect was strongest at low pH values. The authors also concluded that at high pH values the glutamic acid bridge (in the wild-type enzyme) tends to act as destabilising element due to charge repulsions.

The present invention makes use of strong hydrogen bonds in stabilisation of proteins. Since the strength of hydrogen bonds depends on a mixture of several factors, rules predicting the position and type of an amino acid exchange are required to increase the stability of a protein. The present invention uses the comparison between quantum chemical model systems and the statistical analysis of a large set of protein structures to define rules for the geometry (and environment) of very stable hydrogen bonds. Based on this we were able to identify in the three-dimensional structures of proteins pairs of amino acids having amidic side chains, and to propose that changing these amidic side chains to carboxylic acid side chains so as to form acid-acid pairs would increase the protein stability at low or medium pH. Acid-acid pairs can also be removed in order to stabilise a protein molecule at high pH. Furthermore, since acid-acid pairs have a repulsive interaction at high pH values (pH>7), introducing acid-acid pairs can also be used to introduce pH-dependent molecular switches, which will alter the protein properties, such as binding, at elevated pH values.

### SUMMARY OF THE INVENTION

The present invention describes the engineering of a) pairs of carboxylic acid side chains in proteins to pairs of amide-acid or amide-amide residues and b) vice versa, thus resulting in pH-dependent stabilisation or destabilisation of the protein.

Pairs of carboxylic acid side chains are able to form low barrier hydrogen bonds (LBHB), which are under certain conditions much stronger than “normal” hydrogen bonds. These acid-acid pairs can be introduced into many proteins by replacing pairs of amide-acid or amide-amide residues, which form one of the most frequent types of hydrogen bonds. No other amino acid substitution, which changes the nature of hydrogen bonds, is causing so little geometrical strain to a protein.

Based on an exhaustive geometrical search of the Brookhaven Protein Data Bank (PDB) and quantum chemical calculations we have developed rules to define which residues in a protein are good candidates to be replaced by an acid-acid pair. Because of the strength of LBHBs this will allow better stabilisation of proteins at low or medium pH than using other protein engineering methods. Existing acid-acid pairs can also be removed in order to stabilise proteins at high pH. On the other hand, this destabilisation effect of acid-acid pairs at high pH (pH>7) can be used to introduce pH-dependent molecular switches, which would alter the protein properties at elevated pH values.

A general object of the present invention is thus a method for controlling pH-dependent behaviour of a protein, comprising the steps of identifying in a protein structure at least one pair of amino acid residues, wherein the spatial positions of the amino acids within the pair enable formation of hydrogen bonds with altered pH-behaviour between the side chains of said amino acids, wherein said pair is an acid-acid pair, an amide-acid pair or an amide-amide pair, and replacing in at least one position per protein an acid-acid pair by an amide-acid pair or an amide-amide pair, or an amide-acid pair by an acid-acid pair or an amide-amide pair, or an amide-amide pair by an acid-acid pair or an amide-acid pair.

A specific object of the present invention is the stabilisation of proteins at low and medium pH values by introducing pairs of carboxylic acids, which are hydrogen bonded via their side chains.

Another specific object of the invention is the stabilisation of proteins at high pH values by replacing acid-acid pairs by their corresponding amide-amide or amide-acid pairs.

A further object is to introduce and use pairs of carboxylic acid side chains as pH-dependent molecular switches in proteins.

### BRIEF DESCRIPTION OF THE DRAWINGS

FIG. 1: Definition of the angles used to analyse the geometry of the interacting acid-acid pairs. [distances r₁ (O—H), r₂ (H . . . O), r₃ (O . . . O), angle a₁ between O—H . . . O, dihedral angles d₁ and d₂ between O—C—O . . . O, d₃ the angle between the planes of the carboxyl groups defined as the dihedral (C—O . . . O—C)]

FIG. 2: Schematic representation of the three different spatial arrangements of acid-acid pair structures defined by combinations of dihedral angles d₁ and d₂.

FIGS. 3a and 3b: Distribution of the hydrogen bond angle a₁ (between O—H . . . O) in amide-acid pairs (3a) and acid-acid pairs (3b) in proteins. A Gaussian function is fitted to the data.

FIGS. 4a, 4b and 4c: Distribution of the dihedral angle d₃ in the three different spatial arrangements of d₁ and d₂ of the interacting carboxyl groups in proteins. The minimum from the quantum chemical optimisation is in each case marked with an arrow.

FIG. 5: Melting temperatures of Trichoderma reesei cellobiohydrolase Cel6A (CBHII) wild-type enzyme and two acid pair mutants as a function of pH. Intrinsic tryptophan fluorescence of Cel6A and the mutants was monitored using excitation wavelength=280 nm, emission wavelength=340 nm, and band width=5nm(ex), 5 nm(em). The enzyme concentration was 0.5 μM. Samples were heated gradually up to 80° C. and emission intensity at 340 nm was recorder after every 0.5° C. Data was plotted as a function of temperature, smoothed and differentiated by using the Origin software package. The melting temperatures of the different enzymes were determined by the culmination point of the curves at different pH values. All points (except pH 3) were measured at least as duplicates. Buffers used were: 50 mM citrate/HCl, pH 3.0, 1.4 mS; 50 mM glycine/HCl pH 3.7, 0.3 mS; 50 mM sodium acetate pH 5, 1.9 mS; 50 mM potassium phosphate pH 6, 3.7 mS; 50 mM potassium phosphate pH 7, 5.7 mS; 50 mM potassium phosphate pH 8, 6.9 mS; 50 mM sodium borate/HCl pH 9, 4.8 mS. Cel6A wild-type (□); Cel6A E107Q mutant (∘); Cel6A E107Q/D170N/D366N mutant (⋄).

FIG. 6: Half-lives of cellobiohydrolase Trichoderma reesei Cel6A (CBHII) wild-type and two acid pair mutants at 44° C., pH 7.7. The figure shows a semi-logarithmic plot of the decrease of activity on cellotetraose at 44° C. versus incubation time at pH 7.7. The enzyme was preincubated for indicated time periods at 44° C., pH 7.7, after which the activity on cellotetraose (final conc. of 300 μM) was measured using the same temperature and pH. Activity was normalized to 100% at zero time for each enzyme. Cel6A wild-type (□); Cel6A E107Q mutant (∘); Cel6A E107Q/D170N/D366N mutant (⋄).

FIG. 7a, 7b and 7c: The activity of Trichoderma reesei Cel6A (CBHII) wild-type enzyme and the two acid pair mutants at 44° C., pH 7.5 on bacterial microcrystalline cellulose (BMCC) at three time points (0 h, 2 h and 21 h). The enzyme (1.9 μM enzyme) was preincubated for 0 h (7a=panel A), 2 h (7b=panel B) and 21 h (7c=panel C) at 44° C., pH 7.5, after which the activity on BMCC (final conc. of 0.7 mg/ml) was measured using the same temperature and pH. Cellobiose (Glc₂) was used as a standard and the reducing sugars were measured with PAHBAH reagent (see Example 1 for details). Cel6A wild-type (□); Cel6A E107Q mutant (∘); Cel6A E107Q/D170N/D366N mutant (⋄).

### DETAILED DESCRIPTION OF PREFERRED EMBODIMENTS

The three main ideas of the invention are summarised as follows:

(1) Stabilisation of proteins at low and medium pH values by introducing acid-acid pairs.

(2) Stabilisation of proteins at high pH values by replacing acid-acid pairs.

(3) Pairs of carboxylic acid side chains as pH-dependent molecular switches in proteins.

Possible applications:

Due to the universal occurrence of amide-acid and amide-amide hydrogen bonds in proteins the introduction of acid-acid pairs for the thermal stabilisation would be possible for an enormous amount of different proteins which are used in enzyme-catalysed processes around neutral pH values. There are enzymatic processes at low pH (e.g. with amylases) for which the proteins have to be stabilised. One reason for the inactivation at low pH values is the protonation of carboxylic acids involved in hydrogen bonds. This would be one of the most promising applications for introducing acid-acid pairs in a protein.

Stabilising proteins at high pH values by replacing acid-acid pairs by e.g. amide-acid pairs might be of limited use because acid-acid pairs do not seem to be very frequently found in proteins. However, for the few examples where they exist (e.g. in Trichoderma cellulases) this is a promising and proven approach for this so far unsolved problem.

For instance, as described in Example 1, for the stabilisation of Trichoderma reesei cellobiohydrolase Cel6A at high pH, two mutants thereof turned out to be useful. In the first one the acid-acid pair E107-E399 of T. reesei Cel6A cellulase is replaced by an amide-acid pair Q107-E399 by making a single point mutation E107Q. In the second one three acid-acid pairs E107-E399, D170-E184 and D366-D419 of T. reesei Cel6A cellulase are replaced by three amide-acid pairs Q107-E399, N170-E184 and N366-D419 by making three point mutations E107Q, D170N and D366N.

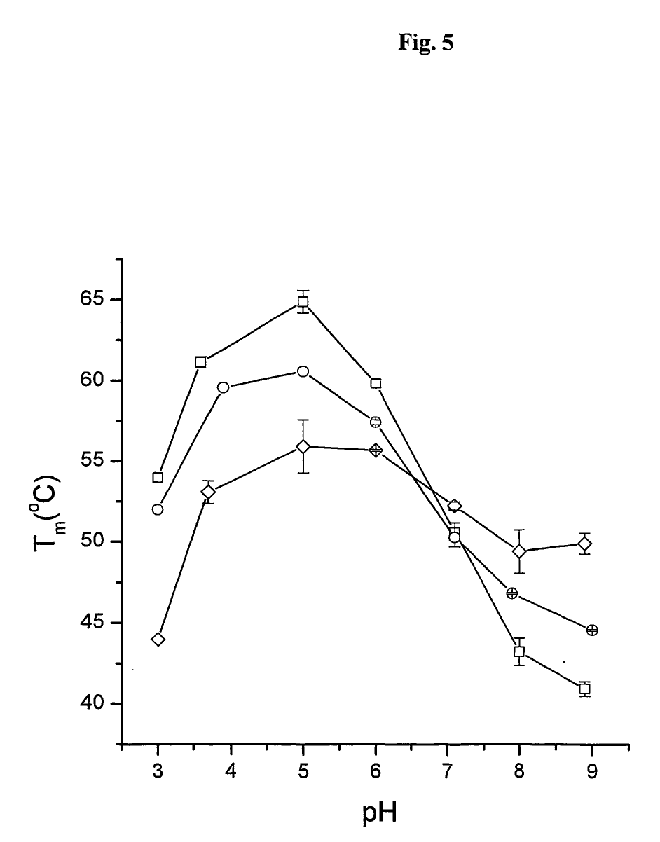

On the other hand, these replacements lead to a decrease of the denaturation temperature of the mutant proteins for about 4° C. per acid-acid pair in the pH range from 3 to 6 as compared to the wild-type enzyme. This result can be seen from FIG. 5.

Due to the side chain repulsion at high pH values, acid-acid pairs could be used as pH-dependent molecular switches either between two protein domains or within one protein domain. A very interesting application is the elution of ligands from an immunoaffinity column containing antibody fragments composed of two or four domains, which bind the ligand very strongly. With acid pairs introduced to the antibody between the two variable domains, harsh elution conditions could be avoided by simply using elevated pH values (>7.5).

In the present specification and claims the expression “pair of carboxylic acid side chains” has the general meaning of a pair of carboxylic acid side chains included in amino acid residues which are hydrogen bonded via their side chains. The expressions (1) “acid-acid pair”, (2) “amide-acid pair” and (3) “amide-amide pair” are used to define the three types of pairs, i.e. those having (1) two carboxylic acid side chains, (2) one amidic side chain and one carboxylic acid side chain, and (3) two amidic side chains. Examples of amino acids involved in such pairs are Asp (D), Asn (N), Glu (E) and Gln (Q).

For the purposes of the present invention, the term “pH-dependent molecular switch” can be defined as a molecular arrangement of at least two functional groups, which are able to change their protonation state. Upon change of the protonation state, an attractive or repulsive interaction between the pair(s) of functional groups will occur. As a consequence of the altered interactions a change in the protein properties or function will occur, such as activation or inactivation of the catalytic function of an enzyme, different ligand/substrate binding modes, or altered stability.

### EXAMPLES

### Theoretical Methods

Statistical Analysis of Protein Structures

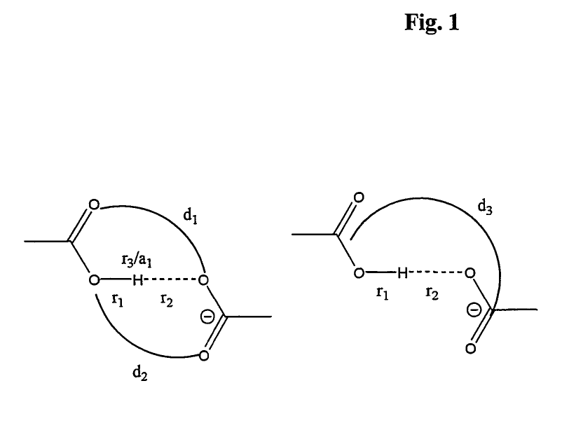

The basis for the statistical analysis was a subset of the PDB (version from Jun. 22, 1999) (Bernstein et al., 1977) composed of 1619 protein structures with less than 90% sequence identity solved by X-ray analysis with an R-factor<0.25 and a resolution better than 2.5 Å. In this subset prepared by Hooft et al. (1996) we were searching for pairs of carboxylic acid oxygens which are in closer contact than 2.9 Å. From these pairs we further excluded those pairs which complex bivalent metal ions thus leading to a short O—O distance. The hydrogen bonding geometry for the resulting 219 pairs was analysed (for an explanation see FIG. 1). For comparison the process was repeated for amide-acid and amide-amide pairs using a distance cut-off of 3.5 Å resulting in 5752 pairs.

For the geometric analysis the CCP4 programs CONTACT, ANGLES and GEOMCALC were used (CCP4, 1994). The angles between the oxygens and the carbonyl carbon were used to calculate the hydrogen bond angle a₁ (FIG. 1). The dihedral angles d₁ and d₂ between oxygen, carbon and the oxygens involved in the hydrogen bond in the first and in the second residue and vice versa were calculated (FIG. 1). This allows a classification according to the lone pairs of the oxygens involved in the hydrogen bond. The dihedral angle d₃ between the carbons and oxygens forming the hydrogen bond (FIG. 1) describes how much the plane of one carboxyl group deviates from the plane of the other.

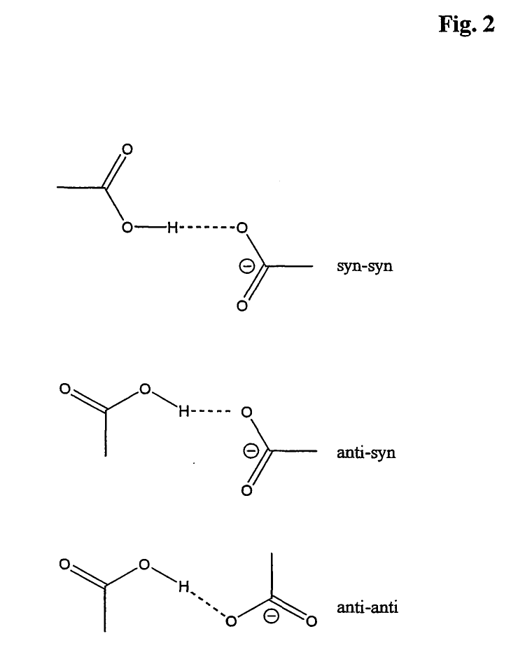

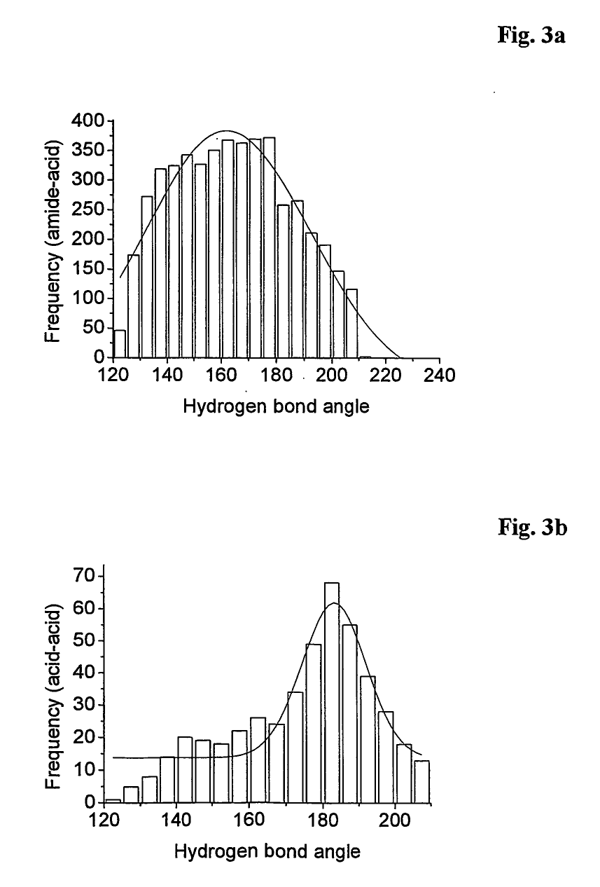

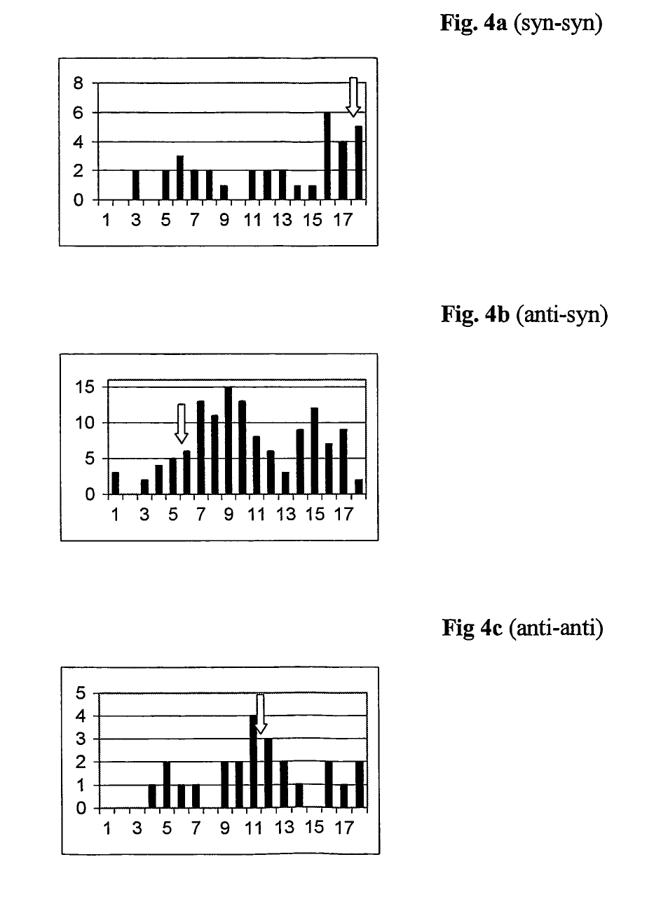

The distribution of O—O distances (r₃, see also FIG. 1) of the acid pairs shows a maximum around 2.55 Å, whereas the maximum for acid-amide pairs is around 2.9 Å. The angles a₁ in acid-acid pairs are distributed around 180° and the distribution is quite narrow. This distribution is much broader for acid-amide pairs suggesting a stronger dependence of acid-acid hydrogen bonds on linear angles (FIGS. 3a and 3b). Three different groups of spatial arrangements (d₁, d₂) were defined via the position of the hydrogen bond relative to the other oxygen of the carboxyl group: (a) syn-syn (120°-180°, 120°-180°), (b) anti-syn, (0°-60°, 120°-180°), and (c) anti-anti (0°-60°, 0°-60°) (FIG. 2). 30 pairs were excluded from further analysis because one of their dihedral angles were between 60° and 120° which makes the classification into one of the geometrical groups uncertain (FIG. 2, Table 1). In the resulting 189 pairs the anti-syn arrangement is with 68% the most common and syn-syn (13%) the least frequent. In the different geometrical arrangements certain orientations of the carboxyl planes relative to each other are preferred. For anti-anti arrangement this is around 120°, for anti-syn around 90° and for syn-syn around 170° (FIGS. 4a to 4c).

The surface accessibility of side chain oxygen atoms of the residues forming hydrogen bonds was calculated with the method of Lee and Richards (1971) implemented in the program PSA (Mizuguchi et al., 1998) using a probe radius of 1.4 Å. 49% of the side chain oxygens in the acid-acid pairs residues show a solvent accessibility of less than 1 Å², which means that they probably do not interact with water. Only 7% of the oxygens have a solvent accessibility over 10 Å², which means that two water molecules interact with a single oxygen in an acid-acid pair hardly ever. The accessibility is quite often nearly equal for both oxygens which hints to a symmetric substitution.

Quantum Chemical Geometries and Energies

In order to explain the distribution of hydrogen bond geometries found in proteins small molecular models representing each type of spatial arrangement have been built. Acetic acid and acetate molecules were used as model system for the pairs of carboxylic acid side chains observed in proteins. The functionality of these molecules is equivalent to that of aspartate and glutamate side chains. Acetamide was used to mimic asparagine and glutamine. All ab initio calculations were performed with the GAUSSIAN 98 suite of programs (Frisch et al., 1998). The geometry of the structures was initially optimised using the 6-31++G(d,p) basis set on the B3LYP level of theory (Becke, 1993; Lee et al., 1988).

Three different structures corresponding to anti-anti, anti-syn and syn-syn minimum structures could be identified as a result from all different starting geometries (FIG. 2); Table 1 contains their energies and geometries. The anti-syn system shows the lowest energy and anti-anti is only 0.4 kcal higher and the syn-syn configuration lies 2.0 kcal above. The syn-syn system has the shortest O—O distance and the hydrogen is more centred there. The results from the quantum chemical calculations match remarkably well with the observed geometries in protein structures (FIGS. 4a to 4c). We conclude that some of the energetic features from the gas phase calculations can also be transferred to the protein environment.

Rules for Strong Hydrogen Bonds in Proteins

Combining the ideas from the statistical analysis of X-ray structures and from the quantum chemical calculations, the following structural constraints for amino acid substitutions were developed. The more of these criteria are fulfilled, the stronger a hydrogen bond between the pair of side chains should be, thus leading to increased stability of a protein variant:

1. A short distance (<2.7 Å) between the hydrogen donor and acceptor atoms should be possible.

2. An angle close to 180° for the hydrogen bond should be possible.

3. An anti-syn arrangement of the carboxyl groups is preferred.

4. The planes of the carboxyl groups should be around 60° for anti-syn, 120° for syn-syn and 180° for anti-anti.

5. The carboxyl oxygens should have as low solvent accessibility as possible.

6. If carboxyl oxygens are accessible to the solvent, then the interactions should be similar for both acids involved in the hydrogen bond.

Table 1: Calculated energies and minimum geometries for different spatial arrangements of acid-acid and amide-acid pairs using the 6-31++G(d,p) basis set. The anti-anti conformation for the amide-acid pair is not stable without constraints and therefore these values are excluded. The nomenclature for the geometrical terms of the interacting amino acid pairs is explained in FIG. 1.

| | syn-syn | syn-syn | anti-syn | anti-syn | anti-anti |
|---|---|---|---|---|---|
| | (acid-acid) | (amide-acid) | (acid-acid) | (amide-acid) | (acid-acid) |
| E_{HB}^{a} | 0.9 (2.0)^{b} | 0.4 | 0.0 | 0.0 | 0.3 (0.4)^{b} |
| rel. | | | | | |
| r₃ | 2.44 | 2.72 | 2.53 | 2.76 | 2.47 |
| r₁ | 1.13 | 1.05 | 1.04 | 1.05 | 1.08 |
| r₂ | 1.32 | 1.68 | 1.49 | 1.71 | 1.39 |
| a₁ | 172.4° | 171.8° | 171.3° | 174.3° | 178.5° |
| d₁/d₂ | 1.6°/2.0° | 2.3°/5.3° | 179.1°/9.0° | 179.8°/0.7° | 179.9°/ |
| | | | | | 180.0° |
| d₃ | 120.1° | 143.9° | 55.1° | 179.9° | 179.9° |

^{a}For the calculation of the stability of different configurations the zero point vibrational energies (ZPVE) obtained from frequency calculations are included.
^{b}The energy differences in brackets are obtained by geometry optimisation at the B3LYP/6-311++G(3df, 3pd) level.

### Example 1

### Stabilisation of Trichoderma reesei Cellobiohydrolase Cel6A (Formerly CBHII) at High pH Values by Replacing One or Three Acid-acid Pairs by Amide-acid Pairs

Acid-acid pairs can be replaced by pH-independent amide-acid pairs to avoid repulsion of the side chains at higher pH values thus stabilising the protein (at high pH). Based on the rules stated above the crystal structure of Trichoderma reesei cellobiohydrolase Cel6A shows four acid-acid pairs, out of which one is in the active centre of the enzyme (D175-D221), and is involved in the catalysis (Zoo et al., 1999). In order to stabilise the enzyme at high pH, two different mutants of T. reesei cellobiohydrolase Cel6A were constructed. In the first one the acid-acid pair E107-E399 of T. reesei Cel6A cellulase was replaced by an amide-acid pair Q107-E399 by making a single point mutation E107Q. In the second one three acid-acid pairs E107-E399, D170-E184 and D366-D419 of T. reesei Cel6A cellulase were replaced by three amide-acid pairs Q107-E399, N170-E184 and N366-D419 by making three point mutations E107Q, D170N and D366N. The point mutations were introduced to the cloned cDNA of T. reesei Cel6A by PCR overlap extension method and the DNA sequence of the whole mutated area was subjected to DNA sequencing. Mutations E107Q (GAA→CAA) and E107Q/D170N/D366N (GAA→CAA/GAC→AAC/GAC→AAC) were first introduced to the cel6A cDNA in pSP73 plasmid (Promega). E. coli strain DH5α (Promega) was used as the cloning host for all the DNA constructions. The mutated cDNAs were then cloned under the T. reesei cel7 (formerly cbh1) promoter of the fungal expression construction as described earlier (Koivula et al., 1996a).

T. reesei strain lacking the genes for the endoglucanase Cel5A (EGI) and the cellobiohydrolase Cel6A (CBHII) was used as the production host of the mutant enzymes. For selection of Trichoderma transformants hygromycin selection plasmid pRLMₑₓ30 was used (Mach et al., 1994). Transformation and choosing the best producing transformant was performed basically as described by St\dot{a}hlberg et al. (1996).

The best producing Cel6A mutant strains were grown as described by Srisodsuk et al. (1993) and the mutated proteins were purified essentially as described by Reinikainen et al. (1995). Culture supernatants were separated from mycelia by centrifugation and clarified further by filtration. Sodium azide, phenylmethylsulphonyl fluoride and EDTA were added to final concentrations of 0.02%, 30 μM, and 1 mM, respectively, and the solutions were concentrated with Pellicon Laboratory Cell System using a PTG10 membrane (Millipore, Bedford, Mass.). After desalting with Biogel P-6 (Bio-Rad, Cambridge, Mass.), the protein solutions were run through a DEAE-Sepharose fast flow column (Pharmacia, Uppsala, Sweden) equilibrated in 50 mM Sodium acetate buffer (pH 5.6). Analysis of the fractions was performed using 4-methylumbelliferyl-β-D-glucopyranoside (MeUmb(Glc₁)) and MeUmb(Glc)₂ as a substrate as well as running SDS-PAGE (Laemmli, 1970) and Western blot (Towbin et al., 1979). The flow-through fractions containing Cel6A were further purified by thiocellobioside-based affinity chromatography (Tomme et al., 1998). The purity of the mutant preparates were checked and verified by SDS-PAGE and Western blotting. The presence of contaminating cellulolytic activites were ruled out by measuring the activities against MeUmb(Glc₁), MeUmb(Glc)₂ and hydroxyethyl cellulose (HEC) as described earlier (Koivula et al., 1996b). Concentration of purified wild-type and mutated proteins were determined from UV absorbance at 280 nm using molar extinction coefficient, ε=104 000 M⁻¹ cm⁻¹ measured with total amino acid analysis for the Cel6A wild-type enzyme (A. Koivula, unpublished results).

The stabilities of the purified acid pair mutants Cel6A E107Q and Cel6A E107Q/D170N/D366N, and wild-type Cel6A were determined both with fluorescence and circular dichroism spectroscopy measurements. Unfolding studies based on monitoring the intrinsic tryptophan fluorescence of Cel6A and the mutants were performed on a Shimadzu RF-5000 spectrofluorometer (Eftink, 1995). Emission and excitation spectra were recorded with bandwidth of 5 nm on both monochromators. A thermostated cuvette holder connected to a water bath controlled the temperature of the sample solution. Both Guanidine Hydrochloride (Gdn-HCl) and temperature-induced unfolding of the wild-type and the acid pair mutants of Cel6A was measured.

Temperature-induced unfolding was monitored by heating samples gradually up to 80° C. (Eftink, 1995) and measuring the fluorescence intensity. The temperature of sample solution was measured continuously using a Fluke 52 electronic thermometer equipped with K-type thermocouple that was immersed in the solution. Intrinsic fluorescence of samples was recorded after every 0.5° C. by measuring emission at 340 nm using an excitation wavelength of 280 nm. The change in the fluorescence intensity of the sample was plotted as a function of temperature, smoothed and differentiated by using Origin graphics and data analyses software. The culmination point of each curve was taken as a melting temperature. All points were measured at least in duplicate. Buffers were 50 mM citrate (pH 3.0), 50 mM glycine/HCl (pH 3.7), 50 mM sodium acetate (pH 5), 50 mM potassium phosphate (pH 6, 7 and 8) and 50 mM sodium borate/HCl (pH 9). FIG. 5 shows the melting temperatures measured using tryptophan fluorescence of wild-type Cel6A and the acid pair mutants Cel6A E107Q and Cel6A E107Q/D170N/D366N plotted as a function of pH. These stability measurements of T. reesei Cel6A wild-type and acid pair mutants show a higher melting temperature of the mutants at alkaline pH (above pH 7) as compared to the wild-type enzyme. Furthermore, the triple mutant Cel6A E107Q/D170N/D366N is more stable than the single mutant Cel6A E107Q.

This trend of an increased stability of the mutants at alkaline pH compared to the wild-type enzyme was confirmed by guanidine hydrochloride induced unfolding studies and CD measurements. CD measurements were performed on a Jasco J-720 circular dichroism spectrometer equipped with PTC-38WI Peltier type temperature control system which controlled the temperature in the cuvette. Spectra were recorded from 240 to 190 nm using a 1 mm cell and a bandwidth of 1 nm every 5° C. and every 2.5° C. around the melting temperature until a temperature of 80° C. was reached. The measurements were performed at pH 6 and 8. The results show that both of the mutant enzymes have lower melting temperature at pH 6 but higher at pH 8 when compared to the wild-type enzyme (data not shown). Guanidine hydrochloride was also used to measure unfolding of the enzymes at pH 6 and 8 (Pace, 1986). A Gdn-HCl stock solution (6M) in 50 mM potassium phosphate buffer was diluted in 50 mM potassium phosphate buffer resulting in a two dilution series with an increasing amount of Gdn-HCl (0-4 M). The pH of the two series of solutions was adjusted to pH 6 and 8 by adding 5 M NaOH and the concentration of Gdn-HCl in solution was determined by measuring the refractive index as described by Nozaki, 1972. 20 μl of the enzyme solution and 480 μl of Gdn-HCl solution were mixed giving the final enzyme concentration of 1.0 μM. The pH 6 and 8 series were incubated for 24 h at 22° C. and the intrinsic fluorescence of the samples with an increasing amount of Gdn-HCl was measured at excitation wavelength 280 nm and emission wavelength 340 nm. All points of both series were measured at least in duplicate. The fraction of unfolded protein was calculated using the equation fᵤ=(y-yₙ)/(yᵤ-yₙ); where fᵤ is unfolded fraction, y is fluorescence of sample, yₙ is fluorescence of native sample and yᵤ is fluorescence of unfolded sample. The unfolding studies of Cel6A and the acid pair mutants using Gdn-HCl gave similar results as compared to the circular dichroism spectroscopic analysis described above (data not shown).

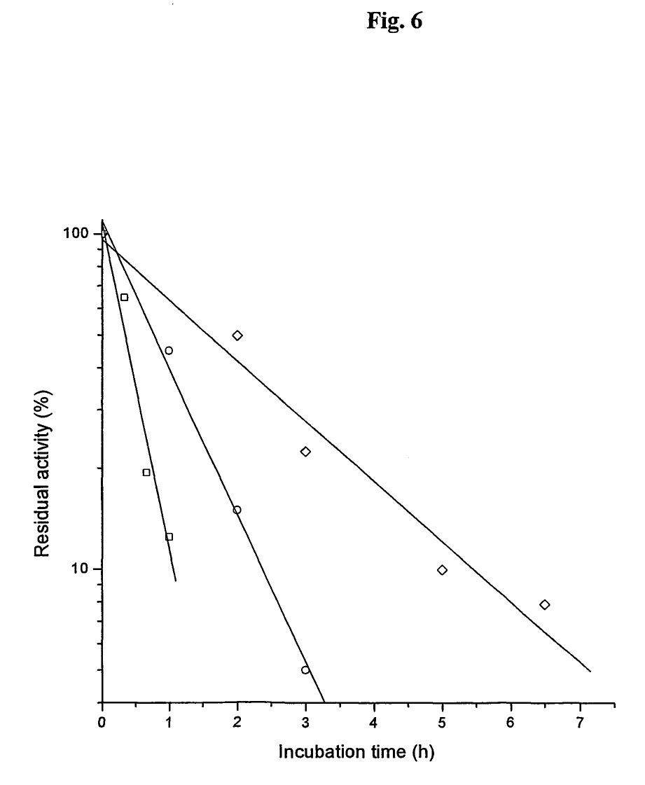

Based on the melting temperature curves shown in FIG. 5, the operational stability of all three enzymes was measured at 44° C. using cellotetraose (Glc₄) as a substrate and varying the pH. Both the incubation and hydrolysis was performed at 44° C. after which the hydrolysis products were analysed by HPLC (Waters Millipore, Milford,. Mass.), equipped with a refractive index detector as described earlier (Teleman et al., 1995). The column used for separation was Aminex HPX-42 A (Bio-Rad) and the deashing cartridges (Bio-Rad) were used before the column in the eluent line to remove buffer ions from the samples. Turnover numbers were obtained from the initial velocities of the reaction curves by a non-linear regression data analysis program (Origin). The cellotetraose concentration in the reaction mixture was 300 μM and the enzyme concentration was between 0.007 μM and 0.15 μM. At pH 5 and 8 activity measurement was also done at substrate concentration 600 μM and 90 μM to verify that 300 μM was the saturating substrate concentration. In all hydrolysis measurements samples were taken at 7-11 different time points, the reaction was stopped and then analysed by HPLC. The following buffers were used: 50 mM citrate/HCl (pH 4), 50 mM sodium acetate (pH 5), 50 mM potassium phosphate (pH 6 and 7) and 30 mM potassium phosphate (pH 8). FIG. 6 shows that the half-life of both of the mutant enzymes Cel6A E107Q and Cel6A E107Q/D170N/D366N at pH 7.7 is increased when cellotetraose is used as a substrate, and that the increase is bigger with the triple mutant E107Q/D170N/D366N.

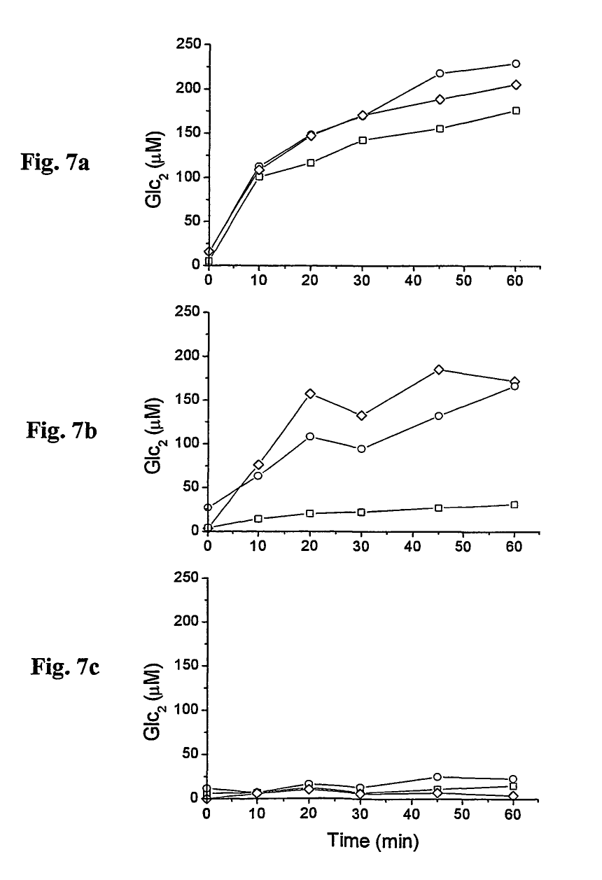

The increased operational stability of both of the mutants was also demonstrated on a more natural substrate, bacterial microcrystalline cellulose. Bacterial microcrystalline cellulose (BMCC) was prepared from Nata de Coco (Reyssons Food Processing, The Philippines) basically as described by Gilkes et al. (1992) resulting in a preparation with DP of 200-300. The enzymatic activity on BMCC was determined by shaking the intact enzymes (final concentration of 1.4 μM and substrate (0.7 mg/ml) at 44° C. in 40 mM sodium acetate, pH 5 and 30 mM potassium phosphate, pH 7.7. Samples were taken at designated time points and the reaction was stopped by adding half the reaction volume of a stop-reagent containing 9 vol. of 94% ethanol and 1 vol. of 1 M glycine, pH 11.2. Samples were filtered through Millex GV 0.22-μm units (Millipore). The soluble sugars were analysed by using p-hydroxybenzoic acid hydrazide as described by Boer et al. (2000). The activity of Cel6A wild-type and the two acid pair mutants on BMCC after three different pre-incubation times is plotted in FIG. 7. As can be seen, both of the acid pair mutants Cel6A E107Q and Cel6A E107Q/D170N/D366N have most of their cellulose degradation activity left after 2 hour pre-incubation at pH 7.7 at 44° C. (FIG. 7, panel B) while the Cel6A wild-type enzyme shows only very low residual activity.

### Example 2

### Pairs of Carboxylic Acid Side Chains as pH-dependent Molecular Switches in Proteins

We have earlier shown that a specific antibody fragment (F_{ab} fragment) called ENA5His can fractionate two enantiomers of a drug (Finrozole) racemate when immobilised in an immunoaffinity column (Nevanen et al., 2001). The results show that one of the enantiomers can be bound specifically to the column in PBS (phosphate buffered saline, pH 7.4), while the other enantiomer elutes in the flow-through. However, to elute the bound enantiomer from the column very high pH values (pH 11.5) are needed, and this high pH causes partial denaturation of the antibody fragment. In order to be able to reuse the immunoaffinity column for many runs, milder elution conditions are required. An acid-acid pair is thus introduced between the two variable domains of the antibody fragment so as to destabilise the orientation of the two domains and consequently also the binding of the enantiomer at high pH. The design of the acid-acid pair E39 (H)-E38 (L) by making mutations Q39E in the heavy chain (H) and Q38E in the light chain (L) of the F_{ab} fragment ENA5His is based on a modelled three-dimensional structure of an antibody-hapten complex. The position numbers are according to the convention of Kabat (Kabat et al., 1987).

The point mutations are made to the cloned cDNA of the antibody fragment ENA5His. ENA5His is an antibody fragment (or F_{ab} fragment) cloned from a monoclonal antibody 95U1 (Nevanen et al., 2001) and composed of two polypeptide chains, i.e. light and heavy chains. Both of these polypeptide chains fold into two domains. A six histidine residue-long tag has been genetically engineered at the C-terminal end of the light chain. Standard methods are used for all recombinant DNA procedures. The plasmid containing the wild-type F_{ab} fragment cDNA is called pENA5His. This construction contains both the heavy and the light chains as a dicistronic operon under the tac promoter controlled by the lacI^{q} repressor present in the expression vector pTI8 (Takkinen et al., 1991). For secretion, the PelB signal sequence of pectate lyase of Erwinia carotovora (Takkinen et al., 1991) is linked in front of both the heavy and the light chain cDNAs. In addition, pENA5His construction contains a six histidine tag added to the C-terminus of the light chain. Mutation Q39E of the heavy chain and Q38E of the light chain are introduced to the cDNA coding for the ENA5His F_{ab} fragment in pENA5His expression plasmid. The resulting mutant construction is called pENA5APHis.

pENA5APHis construction transformed to the Escherichia coli production host is cultivated in a laboratory scale fermenter. The pENA5APHis antibody fragment is purified from the culture supernatant according to standard methods using IMAC (immobilised metal affinity chromatography) and ProteinG affinity chromatography (Nevanen et al., 2001). The purity of the mutant preparate is checked and verified by SDS-PAGE and Western blotting and the concentration of purified wild-type and mutated proteins is determined. The purified mutant protein is immobilised to Chelating Sepharose (Pharmacia) and the column is used to test the fractionation properties of the mutated antibody fragment. The results indicate that the pENA5APHis column is able to fractionate the two enantiomers in a similar manner as the wild-type ENA5His column, but that the elution profile when eluted with a pH gradient of pH 1-14 is altered as compared to the wild-type ENA5His column. Furthermore, the stability of the ENA5APHis mutant protein is tested using tryptophan fluorescence. The unfolding measurements are based on monitoring of intrinsic tryptophan fluorescence at different temperatures at pH range 3.7-9.0. The results indicate the mutant has indeed altered pH behavior at high pH as compared to the wild-type ENA5His F_{ab} fragment.

### References

Becke, A. D. (1993). Density functional thermochemistry. 3. The role of exact exchange. J. Chem. Phys., 98, 5648-5652.

Bernstein, F. C., Koetzle, T. F., Williams, G. J. B., Meyer, E. T., Brice, M. D., Rodgers, J. R, Kennard, O., Shimanouchi, T. & Tasumi, M. (1977). The Protein Data Bank: A computer-based archival file for macromolecular structures. J. Mol. Biol. 112, 535-542.

Boer, H., Teeri, T. T. & Koivula, A. (2000). Characterization of Trichoderma reesei cellobiohydrolase Cel7A secreted from Pichia pastoris using two different promoters. Biotech. Bioeng. 69, 486-494.

Catanzano, F., Graziano, G., Capasso, S. & Barone, G. (1998). Stability of monodeaminated forms of ribonuclease A. In: Stability and Stabilization of Biocatalysts. (Ed.: Ballesteros, A.) Elsevier Science B. V.

CCP4 (1994). Collaborative Computational Project, Number 4. “The CCP4 suite: Programs for protein crystallography”. Acta Cryst. D 50, 760-763.

Eftink, M. R. (1995). Use of multiple spectroscopic methods to monitor equilibrium unfolding of proteins. Methods in Enzymol. 259, 487-512.

Flocco, M. M. & Mowbray, S. L. (1995). Strange bedfellows: Interactions between acidic side-chains in proteins. J. Mol. Biol. 254, 96-105.

Frisch, M. J., G. W. Trucks, H. B. Schlegel, G. E. Scuseria, M. A. Robb, J. R Cheeseman, V. G. Zakrzewski, J. A. Montgomery, Jr., R. E. Stratmann, J. C. Burant, S. Dapprich, J. M. Millam, A. D. Daniels, K. N. Kudin, M. C. Strain, O. Farkas, J. Tomasi, V. Barone, M. Cossi, R Cammi, B. Mennucci, C. Pomelli, C. Adamo, S. Clifford, J. Ochterski, G. A. Petersson, P. Y. Ayala, Q. Cui, K. Morokuma, D. K Malick, A. D. Rabuck, K. Raghavachari, J. B. Foresman, J. Cioslowski, J. V. Ortiz, B. B. Stefanov, G. Liu, A. Liashenko, P. Piskorz, I. Komaromi, R. Gomperts, R. L. Martin, D. J. Fox, T. Keith, M. A. Al-Laham, C. Y. Peng, A. Nanayakkara, C. Gonzalez, M. Challacombe, P. M. W. Gill, B. Johnson, W. Chen, M. W. Wong, J. L. Andres, C. Gonzalez, M. Head-Gordon, E. S. Replogle & J. A. Pople, Gaussian 98, Revision A.5, Gaussian, Inc., Pittsburgh Pa., (1998).

Gerlt, J. A. & Gassman, P. G. (1993). Understanding the rates of certain enzyme-catalyzed reactions: proton abstraction from carbon acids, acyl-transfer reactions, and displacement reactions of phosphodiesters. Biochemistry 32, 11943-52.

Gilkes, N. R, Jervis, E., Henrissat, B., Tekant, B., Miller Jr., R, Warren, A. & Kilburn, D. G. (1992). J. Biol. Chem. 26, 6743-6749.

Hodel, A., Kautz, R. A., Adelman, D. M. & Fox, R. O. (1994). The importance of anchorage in determining a strained protein loop conformation. Protein Science 3, 549-556.

Hooft, R. W. W., Sander, C. & Vriend, G. (1996). Verification of protein structures: Side-chain planarity. J. Appl. Cryst. 29, 714-716.

Kabat, E. A., Wu, T. T., Reid-Miller, M., Perry, H. M. & Gottesman, K. S. (1987). Sequences of proteins of immunological interest. 4th ed., National Institute of Health, Bethesda, Md.

Koivula, A, Reinikainen, T., Ruohonen, L., Valkeajärvi, A, Claeyssens, M, Teleman, O., Kleywegt, G., Szardenings, M., Rouvinen, J., Jones, T. A. & Teeri, T. T. (1996a). The active site of Trichoderma reesei cellobiohydrolase II: the role of tyrosine-169. Protein Eng. 9, 691-699.

Koivula, A., Lappalainen, A., Virtanen, S., Mäntylä, A. L., Suominen, P. & Teeri, T. T. (1996b). Immunoaffinity chromatographic purification of cellobiohydrolase II mutants from Trichoderma reesei strains devoid of major endoglucanase genes. Protein Expr. Purfic. 8, 391-400.

Kumar, A. G. & McAllister, M. A. (1998). Characterization of low-barrier hydrogen bonds. 8. Substituent effects on the strength and geometry of the formic acid-formate anion model system. An ab initio and DFT investigation. J. Am. Chem. Soc. 120, 3159-3165.

Laemmli, U. K. (1970). Nature, 227, 680-685.

Lee, B. & Richards, F. M. (1971). The interpretation of protein structures: estimation of static accessibility. J. Mol. Biol. 55, 379-400.

Lee, C., Yang, G. & Parr, R. G. (1988). Development of the Colle-Salvetti correlation energy formula into a functional of the electron density. Physical Review B 37, 785-789.

Mach, R. L, Schindler, M. & Kubicek, C. P. (1994). Transformation of Trichoderma reesei based on hygromycin B resistance using homologous expression signals. Curr. Genet. 6, 567-570.

Mizuguchi K., Deane, C. M., Blundell, T. L., Johnson, M. S. & Overington, J. P. (1998). JOY: protein sequence-structure representation and analysis. Bioinformatics 14, 617-623.

Mortensen, U. H. & Breddam, K. (1994). A conserved glutamic acid bridge in serine carboxypeptidases, belonging to the α/β hydrolase fold, acts as a pH-dependent protein-stabilizing element. Protein Science 3, 838-842.

Nevanen, T. K., Söderholm, L., Kukkonen, K., Suortti, T., Teerinen, T., Linder, M., Söderlund, H. & Teeri, T. T. (2001). Efficient enantioselective separation of drug enantiomers by immobilised antibody fragments. J. Chromatogr. A, 925, 89-97.

Nozaki (1972). The Preparation of Guanidine Hydrochloride. Methods in Enzymology 26, 43-50.

Pace, C. N. (1986). Methods in Enzymol. 131, 266-280.

Pan, Y. & McAllister, M. A. (1997). Characterization of low-barrier hydrogen bonds. 1. Microsolvation effects. An ab initio and DFT investigation. J. Am. Chem. Soc. 119, 7561-7566.

Reinikainen, T., Henrikson, K., Siika-aho, M., Teleman, O., & Poutanen, K. (1995). Enzyme Microbiol. Technol., 17, 888-892.

Sawyer, L. & James, M. N. G. (1982). Carboxyl-carboxylate interactions in proteins. Nature 295, 79-80.

Smallwood, C. J. & McAllister, M. A. (1997). Characterization of low-barrier hydrogen bonds. 7. Relationship between strength and geometry of short-strong hydrogen bonds. The formic acid-formate anion model system. An ab initio and DFT investigation. J. Am. Chem. Soc. 119, 11277-11281.

Srisodsuk, M., Reinikainen, T., Penttilä, M. & Teeri T. (1993). J. Biol. Chem., 268, 20756-20761.

St\dot{a}hlberg, J., Divne, C., Koivula, A., Piens, K, Claeyssens, M., Teeri, T. T. & Jones, T. A. (1996). Activity studies and crystal structures of catalytically deficient mutants of cellobiohydrolase I from Trichoderma reesei. J. Mol. Biol. 264, 337-349.

Takkinen, K., Laukkanen, M-L., Sizman, D., Alfthan, K., Immonen, T., Vanne, L., Kaartinen, M., Knowles, J. K. C. & Teeri, T. T. (1991). Protein Eng. 4, 837-841.

Teleman, A., Koivula, A., Reinikainen, T., Valkeajärvi, A, Teeri, T. T., Drakenberg, T. & Teleman, O. (1995). Progress-curve analysis shows that the glucose inhibits the cellotriose hydrolysis catalysed by cellobiohydrolase II from Trichoderma reesei. Eur. J. Biochem. 231, 250-258.

Tomme, P., van Tilbeurgh, H., Petterson, O., van Damme, J., Vandekerckhove, J., Knowles, J., Teeri, T. & Claysens, M. (1998). Eur. J. Biochem. 170, 575-581.

Towbin, H., Staehelin, T. & Gordon, J. (1979). Electrophoretic transfer of proteins from polyacrylamide gels to nitrocellulose sheets: procedure and some applications. Proc. Natl. Acad. Sci. USA, 76, 4350-4354.

Zou, J., Kleywegt, G., St\dot{a}hlberg, J., Driguez, H., Nerinckx, W., Claeyssens, M., Koivula, A., Teeri T. T. & Jones, T. A. (1999). Crystallographic evidence for substrate ring distortion and protein conformational changes during catalysis in cellobiohydrolase Cel6A from Trichoderma reesei. Structure 7, 1035-1045.

1. A method for controlling pH-dependent behaviour of a protein, comprising the steps of
identifying in a protein structure at least one pair of amino acid residues, wherein the spatial positions of the amino acids within the pair enable formation of hydrogen bonds with altered pH-behaviour between the side chains of said amino acids, wherein said pair is an acid-acid pair, an amide-acid pair or an amide-amide pair, and
(1) replacing in at least one position per protein an amide-acid pair or an amide-amide pair by an acid-acid pair, where an introduced pair of carboxylic acid residues fulfils at least the rule that
a short distance (<2.7 Å) between the hydrogen donor and acceptor atoms is possible, and, in addition, optionally at least one of the rules
an angle close to 180° for the hydrogen bond is possible,
an anti-syn arrangement of the carboxyl groups is possible,
the carboxyl oxygens have a low solvent accessibility,
the interplanar angle of the carboxyl groups could be around 60° for anti-syn, 120° for syn-syn and 180° for anti-anti, and
solvent-accessible carboxyl oxygens have similar interactions in both acids involved in the hydrogen bond, or
(2) replacing in at least one position per protein an acid-acid pair by an amide-acid pair or amide-amide pair.

2. The method according to claim 1, wherein the hydrogen bonds between the side chains of said pair of amino acids are low barrier hydrogen bonds (LBHB).

3. The method according to claim 1, wherein at least one glutamic acid residue is replaced with a glutamine residue.

4. The method according to claim 1, wherein at least one aspartic acid residue is replaced with an asparagine residue.

5. The method according to claim 1, wherein at least one glutamine residue is replaced with a glutamic acid residue.

6. The method according to claim 1, wherein at least one asparagine residue is replaced with an aspartic acid residue.

7. The method according to claim 1, wherein an amide-acid pair or an amide-amide pair is replaced with an acid-acid pair to form a pH-dependent switch in a protein.

8. The method according to claim 7, wherein the protein is an antibody or a fragment thereof, and the pH-dependent switch controls the affinity of the antibody.

9. The method according to claim 7, wherein the protein is an antibody or a fragment thereof with amides or carboxylic acids in position 39 of the heavy chain, and position 38 of the light chain.

10. The method according to claim 7, wherein the protein structure is ENA5His antibody fragment.

11. The method according to claim 10, wherein the amide-amide pair Q39(H)-Q38(L) is replaced with the acid-acid pair E39(H)-E38(L).

12. The method according to claim 1, wherein an acid-acid pair, which forms a pH-dependent switch in a protein, is replaced by an amide-acid pair or an amide-amide pair in order to decrease the pH-dependency of the protein.
